(19) United States
(12) Patent Application Publication
(10) Pub. No.: US 2017/0105656 A1
(43) Pub. Date: Apr. 20, 2017

(54) BREATH ANALYZER AND BREATH TEST METHOD

(71) Applicant: Anastasia Rigas, Setauket, NY (US)

(72) Inventor: Anastasia Rigas, Setauket, NY (US)

(21) Appl. No.: 15/312,081

(22) PCT Filed: May 22, 2015
(86) PCT No.: PCT/US15/32162

§ 371 Date: Nov. 17, 2016

Related U.S. Application Data
(60) Provisional application No. 62/001,391, filed on May 21, 2014.
(60) Provisional application No. 62/064,251, filed on Oct. 15, 2014.

(51) Int. Cl. A61B 5/08; A61B 5/00; A61B 5/097; G01N 27/12; G01N 33/497

(52) U.S. Cl. CPC A61B 5/082; G01N 27/126; G01N 33/497; A61B 5/097; A61B 5/42; G01N 2033/4975

(57) ABSTRACT

The present invention provides an improved breath analyzer and breath test method to determine the presence of H. pylori in a subject's digestive tract.

### PRIORITY

This application priority to U.S. provisional application Nos. 62/001,832 and 62/013,133, each of which are herein incorporated by reference.

### FIELD

The present application relates generally to a breath analyzer and a breath test method for detecting ammonia in breath to determine presence of H. pylori in a subject's digestive tract.

### BACKGROUND

Helicobacter pylori (“H. pylori”) is a bacteria found in the digestive tract, often in the stomach. More than 50% of the world's population harbor H. pylori in their digestive tract and are considered to have H. pylori infection. Most people contract H. pylori infection during childhood and will never have any signs or symptoms. However, when signs or symptoms do occur with H. pylori infection, they may include a burning pain in the abdomen, nausea, vomiting, frequent burping, bloating and weight loss. H. pylori infection also can cause people to have an increased risk for developing ulcers and certain types of stomach cancer. H. pylori infections can be treated with antibiotics and proton pump inhibitors.

Current diagnostic methods for detecting H. pylori infection have several drawbacks. Existing tests include (a) serological testing to detect anti H. pylori antibodies in blood, (b) obtaining biopsies during an upper gastrointestinal endoscopy and performing rapid urease testing, (c) culturing H. pylori to perform antimicrobial susceptibility testing, and (d) detecting H. pylori antigens in stool. However, these tests are either invasive or are time consuming and cumbersome to complete. Further, several tests are not highly reliable because they have suboptimal performance.

Urea breath tests have also been used. H. pylori converts urea to carbon dioxide and ammonia using its enzyme urease. H. pylori produces large amounts of urease which often comprises about five percent of its total protein. In a urea breath test, a subject undergoes a fasting and then breathes a baseline breath sample into a first bag. The subject then ingests a labeled urea substrate (e.g., a ¹³C or ¹⁴C labeled urea substrate). After a time period, the subject breathes a post-urea breath sample into a second bag. The post-urea breath sample includes expired ¹³CO₂ or ¹⁴CO₂ in the breath. The first bag and the second bag are sent to a lab for an analysis of the level of ¹³CO₂ or ¹⁴CO₂. If the level is above a certain number, the subject is considered positive for H. pylori.

Known urea breath tests also have several drawbacks. First, they use a urea substrate labeled with ¹³C or ¹⁴C. Urea labeled with ¹³C is very expensive. Also, urea labeled with ¹⁴C is undesirable because ¹⁴C is radioactive and cannot be used in children or women of childbearing age. Further, the breath samples must be sent offsite to a lab, which adds cost and inconvenience.

Even further, the offsite lab uses expensive instruments to analyze ¹³CO₂ or ¹⁴CO₂ levels.

Since most people with H. pylori infection never develop symptoms, they would unlikely undergo existing diagnostic procedures because they are invasive, cumbersome or costly. Thus, it would be desirable to provide easy and reliable point-of-care diagnostic test to determine presence of H. pylori infection. It would also be desirable to provide an improved breath test that is less costly, less cumbersome and more convenient than existing tests.

### BRIEF DESCRIPTION OF DRAWINGS

The following drawings are illustrative of particular examples of the present invention and therefore do not limit the scope of the invention. The drawings are not to scale (unless so stated) and are intended for use in conjunction with the explanations in the following detailed description. Examples of the present invention will hereinafter be described in conjunction with the appended drawings, wherein like numerals denote like elements.

FIG. 1 illustrates an acid-base (emeraldine salt (ES)-emeraldine base (EB)) transition for polyaniline;

FIG. 2 shows an embodiment of an ammonia sensor;

FIG. 3A shows the output of the sensor as change in resistance of the polyaniline from exposure to dry nitrogen, and FIG. 3B shows the corresponding change in the current;

FIG. 4 shows the calibration plot of dry nitrogen (ΔR/Ri vs % dry nitrogen concentration) on the sensor using a gas diluter;

FIG. 5A shows the output of the sensor as change in resistance of the polyaniline from exposure to air, and FIG. 5B shows the corresponding change in the current;

FIG. 6A shows the output of the sensor as change in resistance of the polyaniline from exposure to carbon dioxide, and FIG. 6B shows the corresponding change in the current;

FIG. 7 shows the calibration plot of carbon dioxide/air mixture (ΔR/Ri vs % carbon dioxide/air mixture concentration) on the sensor using a gas diluter;

FIG. 8 shows the response of the sensor to carbon dioxide and ammonia after using an sodium hydroxide filter;

FIG. 9A shows the output of the sensor as change in resistance of the polyaniline from exposure to ammonia, and FIG. 9B shows the corresponding change in the current;

FIG. 10 shows the calibration plot of ammonia (ΔR/Ri vs % ammonia concentration) on the sensor using a gas diluter;

FIG. 11A shows the output of the sensor as change in resistance of the polyaniline from exposure to an ammonia/air mixture, and FIG. 11B shows the corresponding change in the current;

FIG. 12 shows the calibration plot for ammonia (ΔR/Ri vs % ammonia concentration) on the sensor using a 20 cc syringe;

FIG. 13 shows overall sensitivities of the sensor to ammonia, carbon dioxide, dry nitrogen and air;

FIGS. 14-15 show the results of testing different clinical samples of exhausted breath;

FIG. 16 shows a schematic of an embodiment of a breath analyzer;

FIG. 17 shows an embodiment of a breath analyzer with a mouthpiece and a main body in a detached configuration;

FIG. 18 shows an embodiment of a breath analyzer with a mouthpiece and a main body in an attached configuration;

FIG. 19 shows an embodiment of an electrical schematic for the breath analyzer;

FIG. 20 shows an embodiment of a desiccant assembly; and

FIG. 21 shows another embodiment of a desiccant assembly.

### SUMMARY

The present invention provides a breath analyzer and breath test method to determine the presence of H. pylori in a subject's digestive tract.

Some embodiments provide a breath analyzer including a sensor, a processor and an electrical circuit that electrically connects the sensor to the processor. In some cases, the sensor comprises an ammonia selective material and a conductive material, wherein the ammonia selective material contacts the conductive material, wherein the ammonia selective material has a resistivity that increases in response to increased concentration of ammonia. The processor detects resistivity in the electrical circuit and uses the resistivity to calculate a concentration of ammonia in the breath sample. The conductive material can include a plurality of electrodes, e.g. interdigitated finger electrodes.

Some embodiments provide a handheld, portable breath analyzer including a removable mouthpiece and a main body. The removable mouthpiece comprises a first portion and a second portion. The first portion is sized and shaped to accommodate a user's lips and the second portion is sized and shaped to removably attach to the main body. The main body includes a sensor, a processor and an electrical circuit. The sensor comprises ammonia selective material that has a resistivity that increases in response to increased concentration of ammonia (e.g., polyaniline is doped with a dopant that increases pH sensitivity of the polyaniline). The electrical circuit operably connects the sensor to the processor and the processor detects resistivity of the sensor and uses the resistivity to calculate a concentration of ammonia.

Some embodiments provide a breath test method used in connection with a portable breath analyzer. The method includes steps of: (a) providing a portable breath analyzer, (b) prompting a subject to exhale a baseline breath sample into a removable mouthpiece, (c) allowing a processor to measure a resistivity of the sensor that occurs when the baseline breath sample contacts the sensor, (d) prompting a subject to exhale a post-urea breath sample into the removable mouthpiece, (e) allowing the processor to measure a resistivity of the sensor that occurs when the post-urea breath sample contacts the sensor, and (f) comparing the measured resistivity of the baseline breath sample to the measured resistivity of the post-urea breath sample.

Some embodiments provide a method of detecting presence of H. pylori in a digestive tract of a subject, the method comprising collecting a baseline breath sample from a subject, determining an amount of ammonia present in the baseline breath sample, collecting a post-urea breath sample from the subject, determining an amount of ammonia present in the post-urea breath sample, and designating a presence of H. pylori in the digestive tract if the amount of ammonia present in the post-urea breath sample exceeds the amount of ammonia present in the baseline breath sample by a predetermined value. In some cases, the collecting a baseline breath sample from a subject and the collecting a post-urea breath sample from the subject comprises collecting both the baseline breath sample and the post-urea breath sample from a single portable breath analyzer. In some cases, the determining an amount of ammonia present in the baseline breath sample comprises exposing the baseline breath sample to a sensor having a resistivity that increases in response to increased presence of ammonia and measuring resistivity of the baseline breath sample, and wherein the determining a concentration of ammonia present in the post-urea breath sample comprises exposing the post-urea breath sample to the sensor and measuring resistivity of the post-urea breath sample.

### DETAILED DESCRIPTION

The following detailed description is exemplary in nature and is not intended to limit the scope, applicability, or configuration of the invention in any way. Rather, the following description provides some practical illustrations for implementing examples of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives. For example, each time the term “comprising” is used, in alternative embodiments, “comprising” can be replaced with “consisting essentially of” or “consisting of.”

The present invention provides an improved breath analyzer and breath test method to determine the presence of H. pylori in a subject's digestive tract. The improved breath analyzer and breath test is less costly and more convenient than existing methods.

H. pylori uses urease to break down urea into carbon dioxide and ammonia. Both the carbon dioxide and ammonia byproducts are present in exhaled breath. Prior urea breath tests use labeled urea substrates and detect the amount of labeled CO₂ in the breath. In contrast, the present breath analyzer and breath test method detects ammonia in breath using an ammonia detecting sensor.

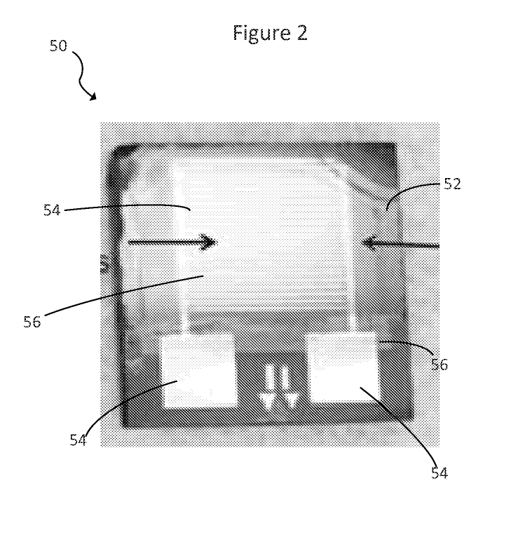

FIG. 2 illustrates an exemplary embodiment of an ammonia detecting sensor 50. The ammonia detecting sensor 50 includes a substrate 52, a conductive material 54 and ammonia selective material 56.

Conductive material 54 is deposited onto a substrate 52. The conductive material 54 includes any desired conductive material. In some cases, the conductive material 54 is platinum. In other cases, the conductive material 54 is gold. In certain cases, the conductive material 54 is an electrode arrangement. The electrode arrangement can be a single electrode or a plurality of electrodes. The electrodes can be spaced apart in any desired arrangement. In some cases, the electrodes are spaced less than about 250 μm apart, perhaps less than about 150 μm apart, such as about 100 μm apart. In certain cases, the electrodes are spaced less than about 10 μm apart, such as 5 μm apart. In some cases, the electrodes include interdigitated finger electrodes. In one embodiment, the sensor comprises interdigitated platinum finger electrodes with a line spacing of 100 μm apart or less. In the embodiment of FIG. 2, the conductive material 54 is provided as an arrangement of interdigitated finger electrodes.

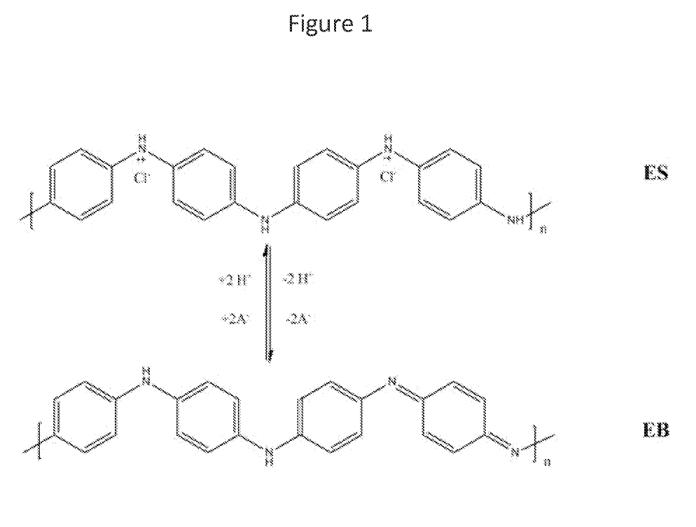

In some cases, the ammonia selective material 56 includes doped polyaniline. Polyaniline exhibits three different oxidation states: leucoemeraldine (LEB, fully reduced), emeraldine (EB, half-oxidized), and pernigraniline (PNB, fully oxidized). Also, when polyaniline is in the emeraldine state, it can be in either an emeraldine salt or emeraldine base form. When in the emeraldine salt form, the polyaniline is conducting. The emeraldine salt form is usually obtained by protonating the basic amine and imine sites with strong acids. This process is reversible in that the emeraldine base form is obtained by deprotonating the amine groups. Thus, the emeraldine state of polyaniline transitions between an acid form and base form. FIG. 1 shows the acid-base transition of polyaniline.

The inventor has discovered that the acid-base transition of polyaniline renders it pH sensitive and this characteristic allows it to be effectively used in ammonia detection. When ammonia contacts an emeraldine salt form of polyaniline, the ammonia deprotonates the amine groups and converts it to an emeraldine base, which also causes an increase in resistivity and a corresponding decrease in conductivity.

Further, the polyaniline can be doped with a protonic acid to increase its pH sensitivity. A polyaniline with increased pH sensitivity is desirable for ammonia detection because when ammonia converts the polyaniline to an emeraldine base, it causes an even larger increase in resistivity and corresponding decrease in conductivity. Larger increases in resistivity (and decreases in conductivity) are desirable because they are easier to detect and increase the sensitivity of the polyaniline to ammonia.

Polyaniline doped with a protonic acid has an increase in pH sensitivity compared to an undoped polyaniline. In some cases, the polyaniline can be doped with a protonic acid including ions such Cl⁻ and SO₄² to obtain pH sensitivity of around 59 mV. Certain protonic acids cause for an even larger increase in pH sensitivity. For example, polyaniline doped with camphor sulfonic acid has been shown to have a pH sensitivity of around 70 mV.

In some cases, the polyaniline comprises at least one dopant that increases pH sensitivity of the polyaniline. In some cases, the dopant is a protonic acid. In some embodiments, the dopant is hydrocholoric acid. In other embodiments, the dopant is camphor sulfonic acid. In yet other embodiments, the dopant is both hydrocholoric acid and camphor sulfonic acid. Other possible dopants include sulfuric acid, salicylic acid, acetic acid, citric acid, tartaric acid, oxalic acid, malonic acid, succinic acid, glutamic acid, adipic acid and phthalic acid. Also, in some cases, the polyaniline has a dopant that provides the polyaniline with a pH sensitivity of more than 59 mV. In one embodiment, the polyaniline has a camphor sulfonic acid dopant, which provides the polyaniline with a pH sensitivity of about 70 mV, which is a higher sensitivity observed than when using other dopants.

Also, the ammonia selective material 56 can be deposited directly onto the conductive material 52 using any desired deposition process. For example, the ammonia selective material 56 can be deposited onto the conductive material 52 using a spin coating method, a chemical vapor deposition method or a sputtering method. In certain cases, the conductive material 52 is coated with spun cast ammonia selective material 56. In certain embodiments, the ammonia selective material 56 is doped polyaniline deposited directly onto the conductive material 52 using a spin coating method. The sensor 50 also includes contact pads 54. The sensor 50 is connected to an electrical circuit via contact pads 54.

The sensor 50 detects ammonia at very low ppb levels. In some cases, the sensor 50 detects breath ammonia at levels lower than 50 ppb (<50 ppb) and as high as 500 ppm, thereby covering all ammonia levels encountered in humans. In certain cases, the sensor 50 detects breath ammonia at levels lower than 50 ppb. In other cases, the sensor 50 detects breath ammonia at levels between about 10 ppb and about 50 ppb.

Certain embodiments provide a breath analyzer that detects presence and concentration of ammonia in a breath sample. The breath analyzer includes an input, a sensor, an electrical circuit and a processor. The input receives a breath sample. The sensor contacts the breath sample. The sensor can have any of the embodiments already described. In some cases, the sensor includes ammonia selective material (e.g., doped polyaniline) and a conductive material. The electrical circuit operable connects to the conductive material to the processor. The processor detects changes in resistivity in the electrical circuit and uses the changes in resistivity to calculate a concentration of ammonia in the breath sample.

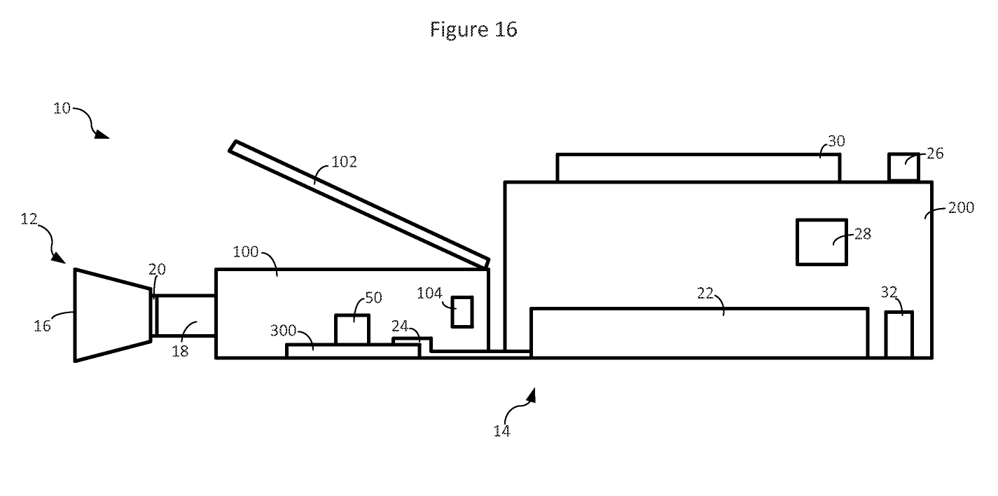

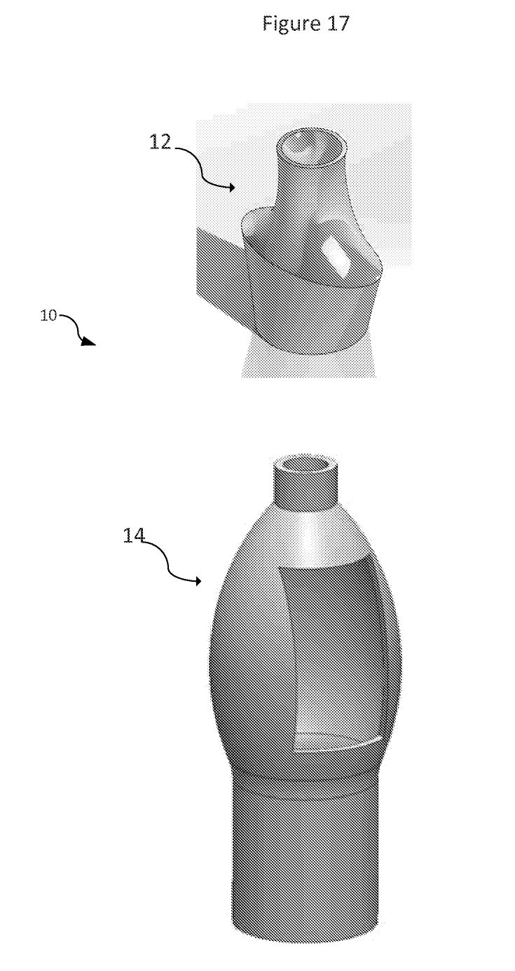

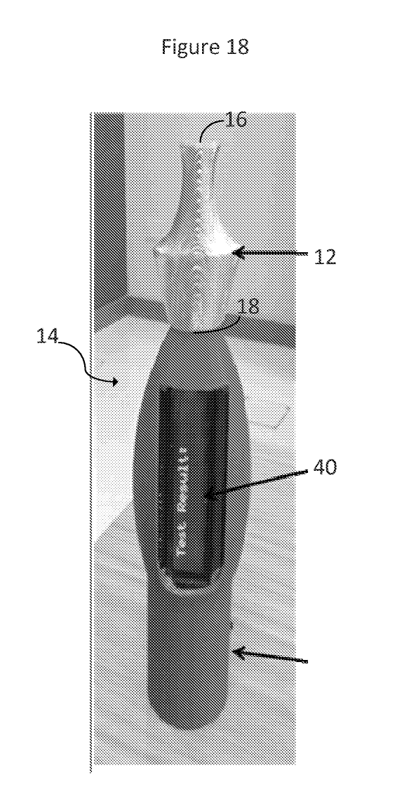

FIG. 16 is a schematic of an exemplary embodiment of a breath analyzer 10. The breath analyzer 10 includes a mouthpiece 12 and a main body 14. FIGS. 17-18 also illustrate an exemplary design of the breath analyzer 10. FIG. 17 shows the breath analyzer 10 with a mouthpiece 12 and main body 14 in an attached configuration whereas FIG. 18 shows the breath analyzer 10 with the mouthpiece 12 attached to the main body 14. The breath analyzer 10 in these embodiments is provided as a self-contained, portable, hand-held device.

The mouthpiece 12 includes a first portion 16 and a second portion 18. The first portion 16 is configured as an input that receives a breath sample. The first portion 16 can be sized and shaped to receive a user's lips, so that a user can blow exhaled breath into the mouthpiece 12. The second portion 18 is sized and shaped to removably connect to the main body 14. For example, the second portion 18 can be snapped onto or perhaps screwed onto the main body 14.

In some cases, the mouthpiece 12 further includes a one-way valve 20. In such cases, a user blows exhaled breath into the mouthpiece 12. The exhaled breath moves forward pass the one-way valve 20 and becomes trapped. In other words, the exhaled breath cannot move backward past the one-way valve 20 and toward the first portion 16.

In certain embodiments, the mouthpiece 12 is a single-use mouthpiece. A single-use mouthpiece is desirable because it can be replaced for use with each new user. Also, in some cases, components of the mouthpiece 12 and/or main body 14 in contact with exhaled breath can be made of an inert or non-reactive material (e.g., polytetrafluoroethylene (PFTE)) that does not interfere with ammonia absorption.

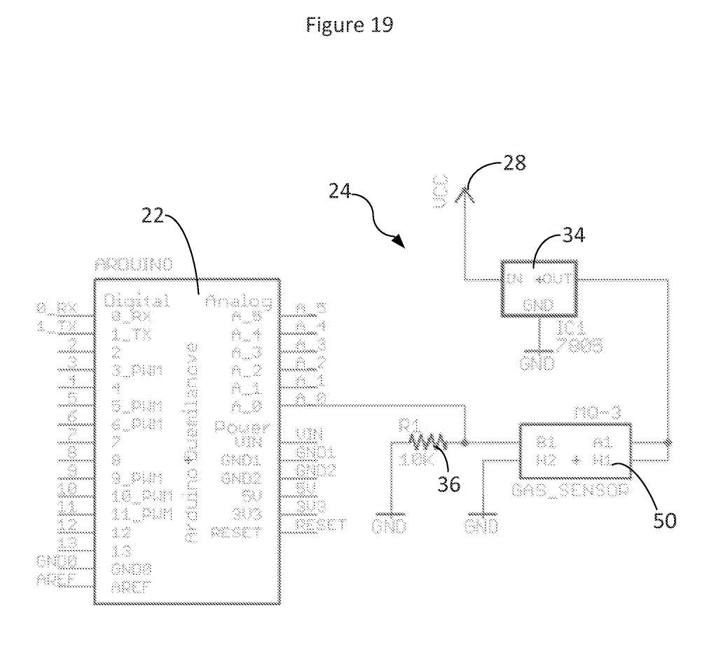

The main body 14 includes a sensor 50, a processor 22 and a power source 28. FIG. 19 is an electrical schematic illustrating the electrical connection between these components according to one embodiment. As shown, the sensor 50, processor 22 and power source 28 are electrically connected via an electrical circuit 24.

The processor 22 can be any desired processor known in the art. In some cases, the processor 22 is a microcontroller. In certain cases, the processor 22 is an Arduino microcontroller.

The sensor can have any of the embodiments already described. In some cases, the sensor includes the sensor 50 of the embodiment of FIG. 2. The sensor 50 is electrically connected to the electrical circuit using any desired connection mechanism. In some cases, the sensor 50 connects to the electrical circuit 24 via an optional sensor mount 300. In such cases, the sensor 50 can be mounted directly onto the sensor mount 300. The sensor mount 300 serves as an interface between the sensor 50 and the electrical circuit 24. Thus, the sensor mount 300 can be any structure known in the art that connects the electrodes 52 of the sensor 50 to the electrical circuit 24. In some embodiments, the sensor mount 300 is a printed circuit board.

In other cases, the sensor 50 is directly connected to the electrical circuit 24. For example, in some embodiments, the electrical circuit 24 includes two metal clips that can be clamped onto the contact pads 54 to create an electrical connection. A user can also replace an old sensor 50 with a new sensor by pulling the old sensor 50 out of the metal clips and inserting a new sensor 50 into the clips.

The main body 14 also includes an on/off button 26 and a power source 28. The power source 28 can be a portable power source, such as a battery. When the on/off button 26 is activated, the power source 28 turns on. As shown in FIG. 19, the power source 28 supplies voltage to a voltage regulator 24. In certain cases, the power source 28 supplies 9 volts to the voltage regulator 24. The voltage regulator 24 regulates the amount of voltage sent to the sensor 50. In some cases, the voltage regulator 24 supplies a voltage to the sensor 50 in the amount of between 0 volts to 5 volts. In certain cases, the voltage regulator 24 supplies a voltage to the sensor 50 in the amount of about 5 volts. In one embodiment, the voltage regulator 24 is an IC1 7805 voltage regulator, a product manufactured by Fairchild Electronics.

A resistor 36 is also electrically connected to the sensor 50 and provides resistance to the sensor. In some case, the resister 36 is a 10 kΩ resistor. When the breath sample contacts the sensor 50, a change in resistivity occurs in the sensor 50 that correlates to an amount of ammonia in the sample. The sensor 50 outputs voltage (along with the changes in resistivity) to the processor 22. The processor 22 detects changes in resistivity in and uses the changes in resistivity to calculate a concentration of ammonia in the breath sample. The processor 22 can also compare a concentration of ammonia between two different breath samples.

The main body 14 can also include a display 30 operably connected to the processor 22. In some cases, the display 30 shows the concentration of ammonia calculated by the processor 22 In other cases, the display 30 shows results of a comparison between a concentration of ammonia between two different breath samples. The comparison results can show a positive comparison result or a negative comparison result that is determined using a predetermined positive/negative threshold. The comparison results can also show a numerical value that is the delta between a baseline ammonia value and a post-urea ingestion ammonia value. The main body 14 can also include an optional wireless connector 32 (e.g., a Bluetooth connector) that transmits data calculated by the processor 22 to an external computer.

In some embodiments, the main body 14 is configured as including a first compartment 100 and a second compartment 200. In some cases, as shown in FIG. 16, the first compartment 100 is a chamber that houses a sensor 50 and sensor mount 300 and the second compartment 200 is an electrical housing that houses various components.

In some cases, the chamber 100 includes a lid or door 102 that opens and shuts. When the door 102 is closed, the chamber 100 provides a closed, sealed environment around the sensor 50. When the door 102 is open, the sensor 50 is accessible through the door opening. A user can open the chamber door 102 to remove and replace the sensor 50 as needed. The chamber 100 also includes an outlet 104. The outlet 104 includes a cap that can be opened to release a breath sample from the chamber 100 and closed to trap a breath sample within the chamber 100.

The mouthpiece 12 is connected to the chamber 100 such that exhaled breath passes from the mouthpiece 12 directly into the chamber 100. The breath analyzer 10 can include an optional desiccant assembly 60. The desiccant assembly 60 helps to remove excess moisture from the exhaled breath. In some embodiments, the desiccant assembly 60 is provided inside of the mouthpiece 12. In other embodiments, the desiccant assembly 60 is provided inside of the chamber 100. Exhaled breath first moves through the desiccant assembly 60 before coming into contact with the sensor 50.

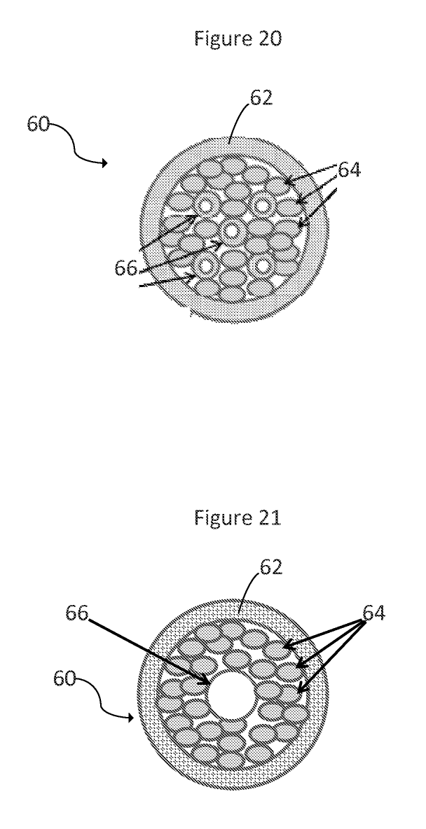

FIG. 20 shows an exemplary embodiment of a desiccant assembly 60. In some cases, the desiccant assembly is provided as a tube 62 upon which exhaled air flows through. The tube 62 can have an interior filled with a plurality of desiccant beads 64. The desiccant beads 64 can also be arranged such that a plurality of channels 66 are created for exhaled air to flow through. FIG. 21 shows another exemplary embodiment of a desiccant assembly 60. In this embodiment, the tube can have an interior filled with a plurality of desiccant beads 64 arranged such that a single channel 66 is created.

Still further, the breath analyzer 10 can include an optional gas filter (not shown). The gas filter helps to remove a selected gas (e.g., carbon dioxide, nitrogen and/or hydrogen) from the exhaled breath. In some embodiments, the gas filter is provided inside of the mouthpiece 12. In other embodiments, the gas filter is provided inside of the chamber 100. Exhaled breath first moves through the gas filter before coming into contact with the sensor 50.

Referring back to FIG. 16, the main body 14 can also include a second compartment configured as an electrical housing 200 that houses various components. In the illustrate embodiment, the electrical housing 200 houses the processor 22, parts of the electrical circuit 24, a power source 28, a display 30, and a wireless connector 32.

During use, a user turns the breath analyzer 10 on by activating the on/off switch 26. The on/off switch 26 can be located anywhere about an exterior surface of the main body 14. This on/off switch 26 in turn prompts the power source 28 to supply voltage to the voltage regulator 34. The voltage regulator 34 regulates and supplies voltage to the sensor 50. The resistor 36 also supplies resistance to the sensor 50.

A user then blows a breath sample into the first portion 16 of the mouthpiece 12. The breath sample moves pass the one way valve 20 and through the optional desiccant/gas filter 60. The breath sample then moves out of the optional desiccant/gas filter 60 and into the chamber 100 where it contacts the sensor 50. The breath sample causes a change in resistivity to occur in the sensor 50. This change in resistivity is outputted to the processor 22.

Other embodiments provide a breath test method. In some cases, the breath test method includes steps of: collecting a baseline breath sample from a subject, determining an amount of ammonia present in the baseline breath sample, prompting the subject to ingest a urea-containing meal (e.g., an unlabeled urea-containing meal), collecting a post-urea breath sample from the subject, exposing the post-urea breath sample to the sensor, determining an amount of ammonia present in the post-urea breath sample, and comparing the amount of ammonia present in the baseline breath sample to the amount of ammonia present in the post-urea breath sample.

In other cases, the breath test method includes steps of: collecting a baseline breath sample from a subject, collecting a post-urea breath sample from the subject, exposing the baseline breath sample to a sensor comprising ammonia selective material having a resistivity that increases in response to increased presence of ammonia, exposing the post-urea breath sample to the sensor, measuring resistivity the baseline breath sample, measuring resistivity of the post-urea breath sample, and comparing the measured resistivity of the baseline breath sample to the measured resistivity of the post-urea breath sample.

The baseline breath sample can include a sample from a subject after fasting. In some cases, the baseline breath sample is a sample from a subject after fasting for at least 2 hours, 3 hours, 4 hours, 5 hours, 6 hours, 7 hours or 8 hours. Also, the post-urea breath sample can include a sample from the same subject after ingesting a urea-containing meal. The baseline breath sample can obtained after fasting and before ingesting the urea-containing material. The post-urea breath sample can be obtained at a set time point after ingesting the urea-containing material. In some cases, the time point is a 15 minute time point. In other cases, the time point is a 30 minute time point.

In some cases, the urea-containing meal is a natural urea-containing meal rather than an isotope urea-containing meal. In certain cases, the urea containing material is a liquid urea-containing meal (e.g., urea powder diluted in liquid). Also, in some embodiments, the urea-containing meal is an unlabeled urea-containing meal. In other embodiments, the urea-containing meal is a labeled urea-containing meal. For example, the urea containing meal can be labeled with ¹³C in some embodiments.

In some cases, the step of comparing the measured resistivity of the baseline breath sample to the measured resistivity of the post-urea breath sample indicates presence of H. pylori in the subject's digestive tract. For example, the method can include determining a positive or negative presence of H. pylori in the subject's digestive tract after comparing the measured resistivity of the baseline breath sample to the measured resistivity of the unlabeled post-urea breath sample. In some cases, the determining a positive presence of H. pylori in the subject's digestive tract occurs when the measured resistivity of the unlabeled post-urea breath sample is greater than the measured resistivity of the baseline breath sample by a predetermined value.

In some cases, the breath test method is performed in connection with a breath analyzer. The breath analyzer can have any of the embodiments described herein. In some cases, the breath analyzer includes a portable, hand-held breath analyzer. In certain cases, the breath analyzer includes a main body and a removable mouthpiece, wherein the removable mouthpiece removably attaches to the main body, wherein the main body includes a sensor, a processor, a power source and an electrical circuit, wherein the electrical circuit operably connects the power source to the sensor and connects the sensor to the processor, wherein the sensor comprises a conductive material and hydrogen selective material in contact with the conductive material, wherein the hydrogen selective material has a resistivity that increases in response to increased concentration of hydrogen.

The breath test method can include steps of: providing a breath analyzer having a sensor comprising ammonia selective material having a resistivity that increases in response to increased presence of ammonia, prompting a subject to exhale a breath sample into the breath analyzer and allowing the breath analyzer to measure a resistivity of the sensor. In certain cases, the breath test method includes steps of: providing a breath analyzer having a sensor comprising ammonia selective material having a resistivity that increases in response to increased presence of ammonia, prompting a subject to exhale a baseline breath sample into the breath analyzer, allowing the breath analyzer to measure a resistivity of the sensor that occurs with the baseline breath sample, resetting the breath analyzer, prompting the subject to ingest a urea-containing meal (e.g., an unlabeled urea-containing meal), prompting a subject to exhale a post-urea breath sample into the breath analyzer, allowing the breath analyzer to measure a resistivity of the sensor that occurs with the post-urea ingestion breath sample and comparing the measured resistivity of the baseline breath sample to the measured resistivity of the post-urea breath sample. The method can also include diagnosing the subject as having an H. pylori infection when the amount of ammonia present in the post-urea breath sample exceeds the amount of ammonia present in the baseline breath sample by a predetermined value.

In some cases, the steps of collecting a baseline breath sample from a subject and collecting an unlabeled post-urea breath sample from the subject comprises collecting both the baseline breath sample and the unlabeled post-urea breath sample from a single portable breath analyzer. Also, in some cases, the step of determining an amount of ammonia present in the a given breath sample can include a step of exposing the baseline breath sample to a sensor comprising ammonia selective material having a resistivity that increases in response to increased presence of ammonia and measuring resistivity of the given breath sample.

Another embodiment of a method of diagnosing an H. pylori infection in a digestive tract of a subject includes steps of: collecting a baseline breath sample from a subject, exposing the baseline breath sample to a sensor comprising ammonia selective material having a resistivity that increases in response to increased presence of ammonia, measuring a resistivity of the sensor that occurs when the baseline breath sample contacts the sensor, prompting the subject to ingest a urea-containing meal (e.g., an unlabeled urea-containing meal), collecting a post-urea breath sample from a subject, exposing the post-urea breath sample to the sensor, measuring a resistivity of the sensor that occurs when the post-urea breath sample contacts the sensor, diagnosing the subject as having an H. pylori infection when the measured resistivity of the post-urea breath sample exceeds the measured resistivity of the baseline breath sample by a predetermined value.

In each of the above breath test methods, the baseline breath sample is a sample from a subject after fasting for at least 2 hours, 3 hours, 4 hours, 5 hours, 6 hours, 7 hours or 8 hours. Also, the step of resetting the breath analyzer comprises flushing the sensor with atmospheric air. In certain cases, the step of flushing the sensor with atmospheric air comprises opening an outlet in the sensor to release the breath sample, allowing the sensor to return to a baseline resistivity and closing the outlet. In other embodiments, the step of resetting the breath analyzer comprises replacing the sensor. In some cases, the step of replacing the sensor occurs each time a breath sample is measured.

### EXAMPLE 1

### Method of Making Sensor

An exemplary method of making a sensor will now be described. The method first included steps of doping an aniline with hydrochloric acid. Double distilled aniline (>99% concentration), hydrochloric acid, ammonium persulfate, ammonium hydroxide (25% concentration), chloroform (>99% concentration) and camphor sulfonic acid (all purchased from Sigma-Aldrich) were obtained. Next, chemical oxidative polymerization of aniline in hydrochloric acid was performed with ammonium persulfate as an oxidant. 50 ml of a solution containing 0.48 M ammonium persulfate in 1 M hydrochloric acid was added to 50 ml of another solution containing 0.4M aniline to create a mixture. This mixture polymerized into a precipitate by resting overnight at room temperature. The precipitate was then collected on a filter paper and washed repeatedly first with 0.1M hydrochloric acid and then with acetone. This resulted in a polyaniline emeraldine salt.

The polyaniline emeraldine salt was then stirred into an aqueous 0.1M NH₄OH solution for 24 hours at room temperature. This resulted in a polyaniline emeraldine base. The polyaniline emeraldine base was then washed with water until the polyaniline obtained a neutral pH. This resulted in a neutral polyaniline doped with hydrochloric acid. The neutralized polyaniline was dried under vacuum for 48 hours at 60° C.

The method next included doping the polyaniline with camphor sulfonic acid. The method included preparing a solution containing 0.5 wt. % of the polyaniline doped with camphor sulfonic acid (e.g., 37.5 mg polyaniline, 48 mg camphor sulfonic acid) in 5 ml chloroform and allowing the solution to dissolve for 2 days with constant stirring. Next, the solution was filtered with a 0.2 μm PTFE syringe filter to remove particular impurities. This resulted in a solution containing polyaniline doped with camphor sulfonic acid.

The method next included depositing the polyaniline doped with camphor sulfonic acid onto a conductive material. Interdigitated platinum film electrodes with a line spacing of 100 μm (purchased from Electronic Design Center at Case Western University) were obtained. The electrodes were cleaned by rinsing them in methanol followed by rinsing them in deionized water and drying them in a stream of dry nitrogen. The electrode pads were also cleaned with a Q-tip dipped in methanol to facilitate direct electrical contact with the analyzer. A spin coating method was then performed to deposit the solution containing polyaniline doped with camphor sulfonic acid onto the electrodes. Specifically, the solution was spun cast onto the electrodes by adding 100 μl of the solution at 500 RPM. This resulted in spun cast polyaniline doped with camphor sulfonic acid deposited on the electrodes.

### EXAMPLE 2

### Breath Analyzer Including the Sensor

One embodiment of a breath analyzer used in each of the Examples will now be described. The breath analyzer included a DropSens flow cell setup (purchased from DropSens, a Spanish company) with a sensor installed in a center of the setup. The analyzer also includes an injector tube connected to the setup. The injector tube was made of a polytetrafluoroethylene (PTFE) material (purchased from duPont), a non-reactive material that does not interfere with ammonia absorption.

During use, a fixed potential of 1V was applied to the electrodes of the sensor. The top of the DropSens flow cell was also closed, thereby sealing the sensor within the cell. Gas samples or breath samples were injected into the flow cell via the injector tube. The sensor's resistivity changed as a function of a gas sample or breath sample flowing over the sensor. The flow cell also included a port that transferred data from the sensor to an Agilent 4155c semiconductor parametric analyzer. Before administering any gas or breath sample, the technician injected air into the analyzer to stabilize and remove moisture from the sensor. Likewise, after injecting any gas or breath sample, the technician again injected air into the analyzer to flush the sensor and return its resistivity to a baseline level.

### EXAMPLE 3

### Effect and Calibration of Dry Nitrogen on Sensor

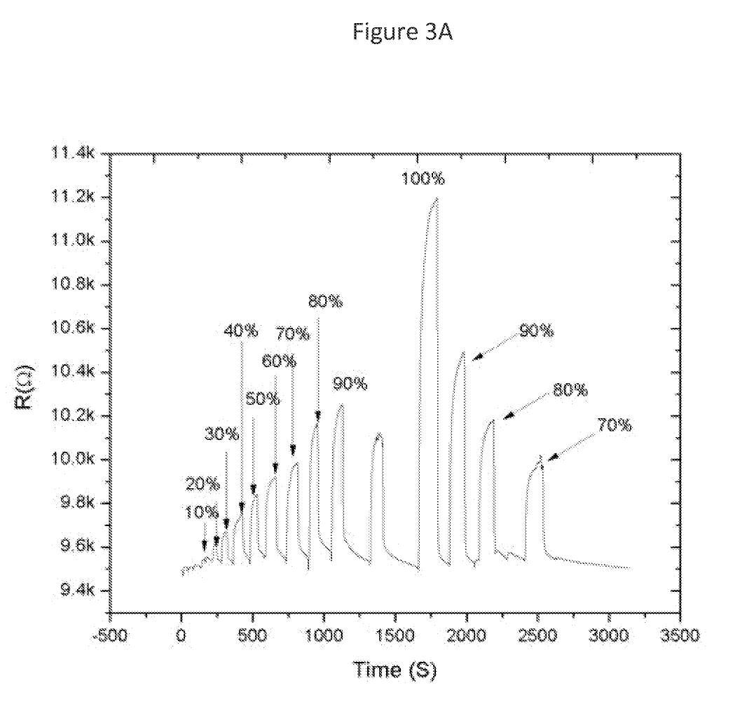

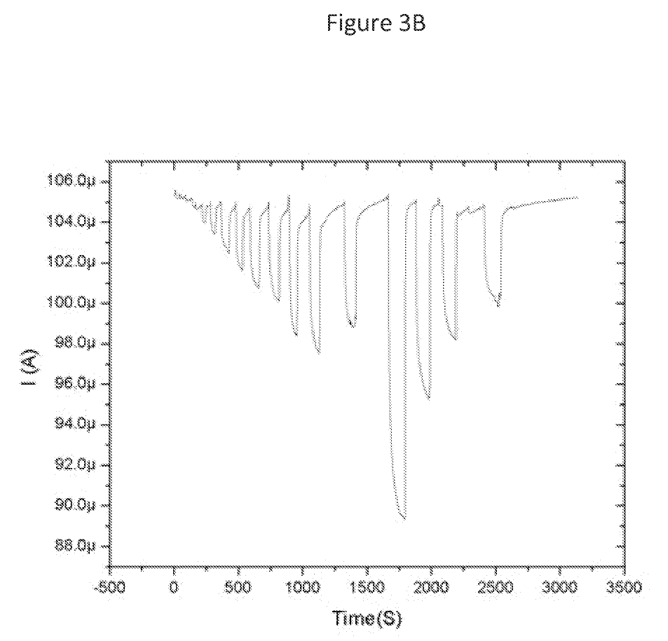

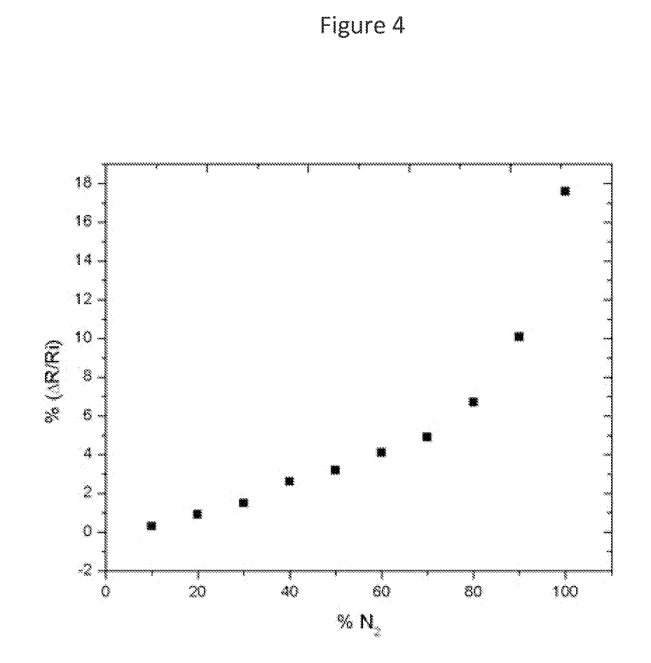

Example 3 was performed to determine the effect of dry nitrogen on the sensor. First, a 5 L Tedlar bag including 25 ppm dry nitrogen calibration gas was connected to an input of a precision gas diluter Model 1010 (purchased from Custom Sensor Solutions). The gas diluter diluted the dry nitrogen with air at dilution percentages from 10-100% and injected the diluted gas into the injection tube of the breath analyzer. At the same time, the resistivity of the sensor was measured for different dilution percentages. FIG. 3A shows the measured resistivity for dilution percentages from 10-100%. FIG. 3B shows the corresponding change in the current. FIG. 4 also shows the response of the sensor plotted as the percentage change in the resistance (ΔR/Ri) vs. the concentration of dry nitrogen, where ΔR=Rf−Ri (Ri being the initial resistance and Rf being the final or observed resistance on exposure to dry nitrogen). A maximum response of about 18% resistivity change was observed with 100% nitrogen. These Figures illustrate that nitrogen alone has an effect on the conductivity of polyaniline, which can be associated with the removal of surface/bulk trapped water molecules in the polyaniline film.

### EXAMPLE 4

### Effect and Calibration of Air on Sensor

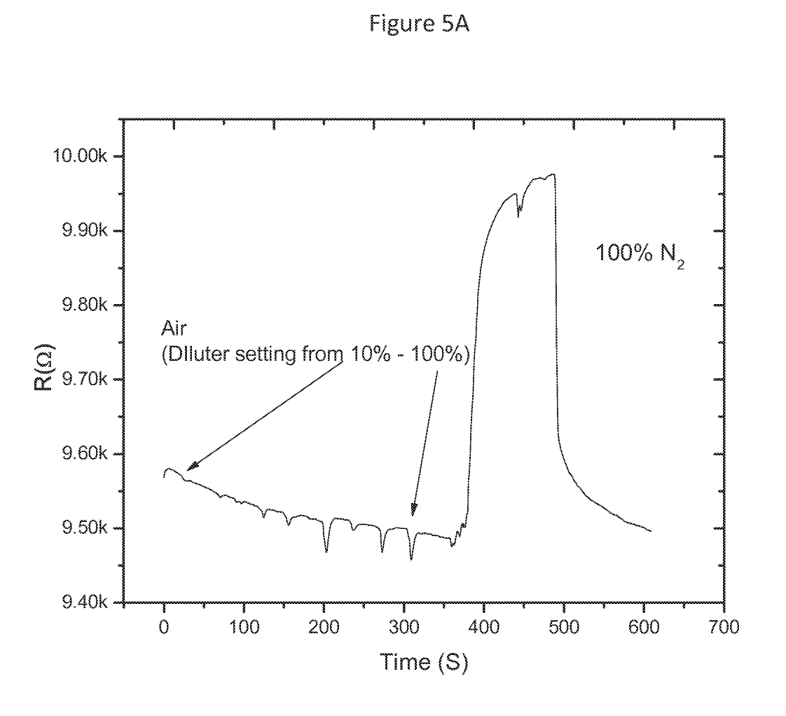

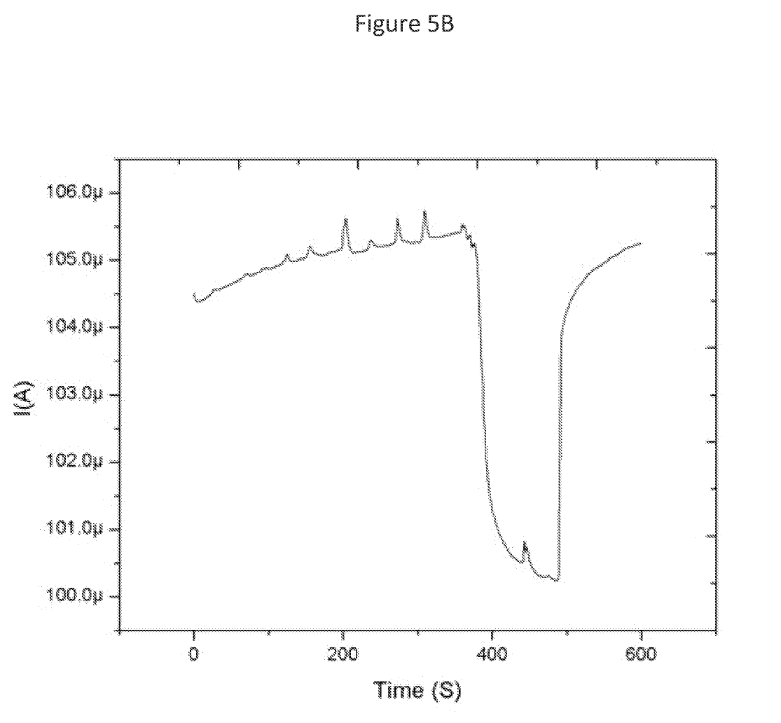

Example 4 was performed to determine the effect of air on the sensor, since air is used to make dilutions in other gas samples. In this case, a separate gas bag was not connected to the gas diluter. Rather, the technician simply left the diluent gas connection open and changed the dilution settings. FIG. 5A shows the measured resistivity for different dilution percentages. FIG. 5B shows the corresponding change in the current. These Figures show that air has no effect on the performance of the sensor. The Figures do show jumps in the sensor responses, but these jumps are due to the perturbations due to opening of the valves when changing the dilution settings on the diluter. To confirm that air has no effect on the sensor, the technician passed 100% dry nitrogen over the sensor and the Figures show that the resistivity increased (and the current dropped) immediately.

### EXAMPLE 5

### Effect and Calibration of Carbon Dioxide on Sensor

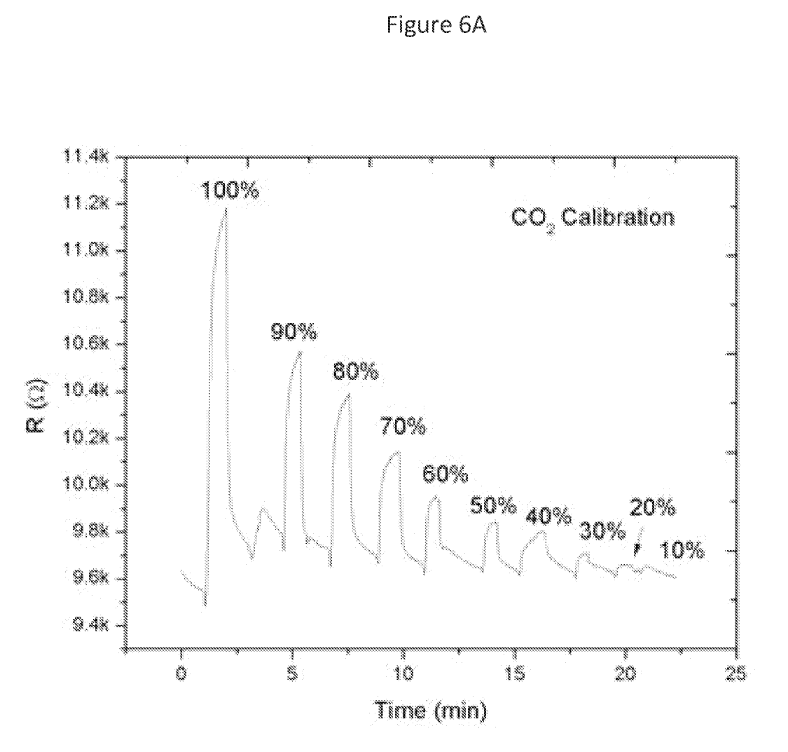

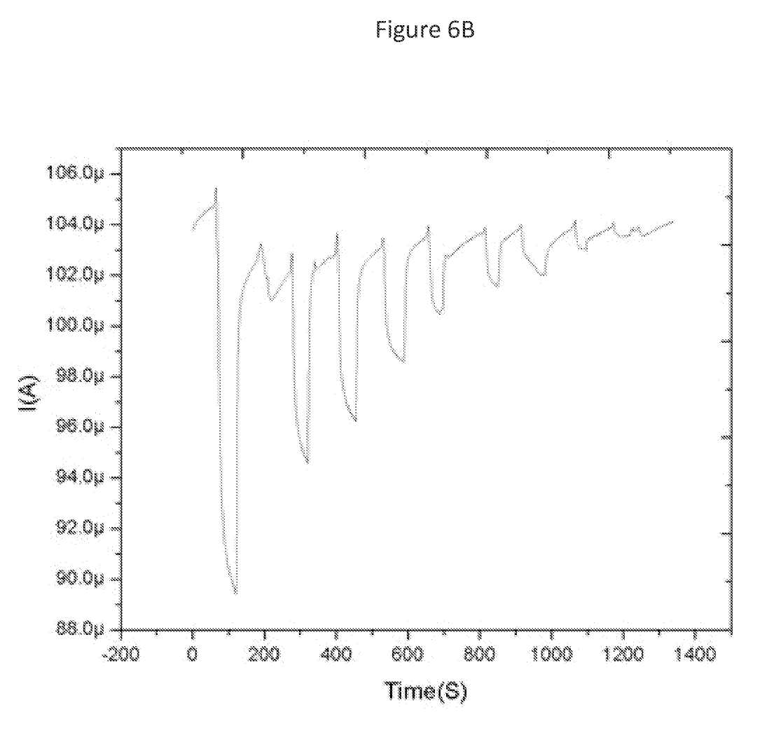

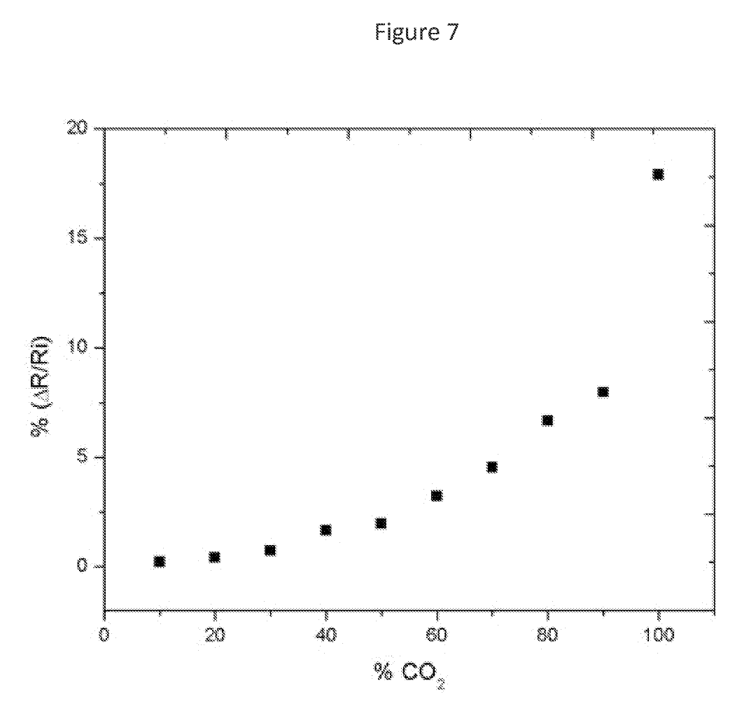

Example 5 was performed to determine the effect of carbon dioxide on the sensor, since 4% of exhaled breath air includes carbon dioxide. First, a 5 L Tedlar bag including carbon dioxide calibration gas was connected to an input of the gas diluter. The gas diluter diluted the carbon dioxide with air at dilution percentages from 10-100% and injected the diluted gas into the injection tube of the breath analyzer. At the same time, the resistivity of the sensor was measured for different dilution percentages. FIG. 6A shows the measured resistivity for dilution percentages from 10-100%. FIG. 6B shows the corresponding change in the current. FIG. 7 also shows the response of the sensor plotted as the percentage change in the resistance (ΔR/Ri) vs. the concentration of carbon dioxide, where ΔR=Rf−Ri (Ri being the initial resistance and Rf being the final or observed resistance on exposure to carbon dioxide). A maximum response of about 18% resistivity change was observed with 100% carbon dioxide. However, a resistivity change of only 0.2% was observed with 10% carbon dioxide. These Figures illustrate that carbon dioxide does have some effect on the conductivity of the sensor although not a considerable one.

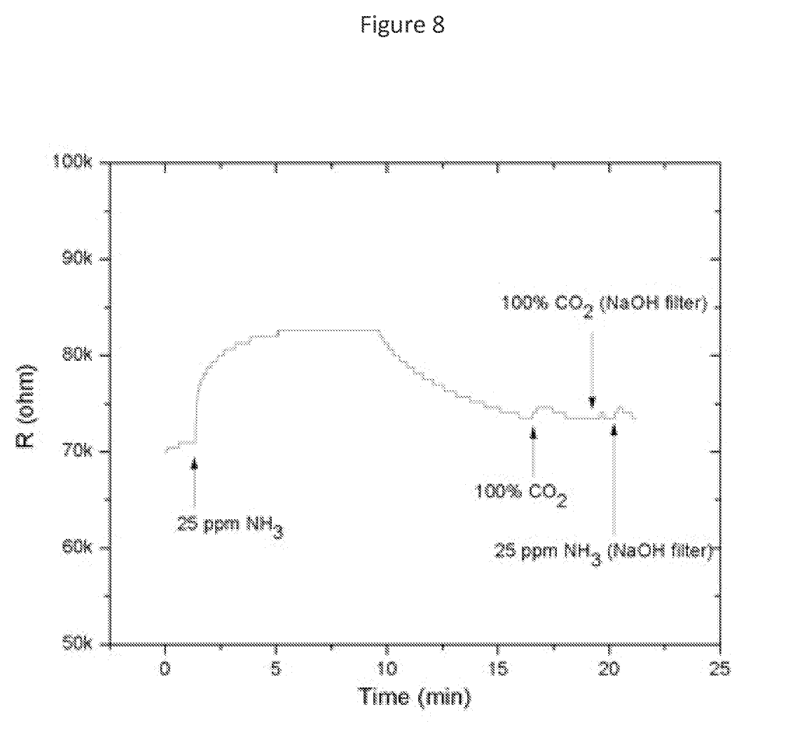

Although there was not a considerable response of the sensor towards carbon dioxide, it might be assumed that the carbon dioxide might interfere while testing for breath ammonia as the levels associated with breath ammonia are at very low ppb levels (e.g., as low as 10 ppb). Therefore, the use of a sodium hydroxide filter to filter out carbon dioxide and humidity from exhaled breath was also investigated. A technician packed Sodium hydroxide into a small 10 cc tube and sealed the ends with septum valves. The technician then passed gas through the Sodium hydroxide filter and recorded its filtering efficiency, as shown in FIG. 8. FIG. 8 shows that the Sodium hydroxide filter effectively absorbs carbon dioxide. In practice, a filter may not be needed for accurate results. However, if used, the size of the filter should be selected so as not to be so large as to also factor out appreciable ammonia.

### EXAMPLE 6

### Effect and Calibration of Ammonia on Sensor

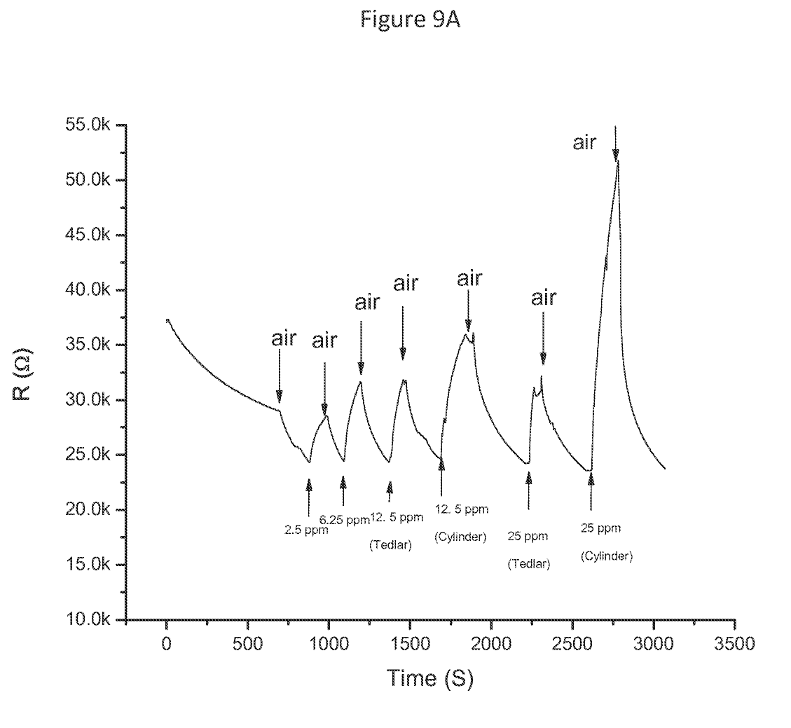

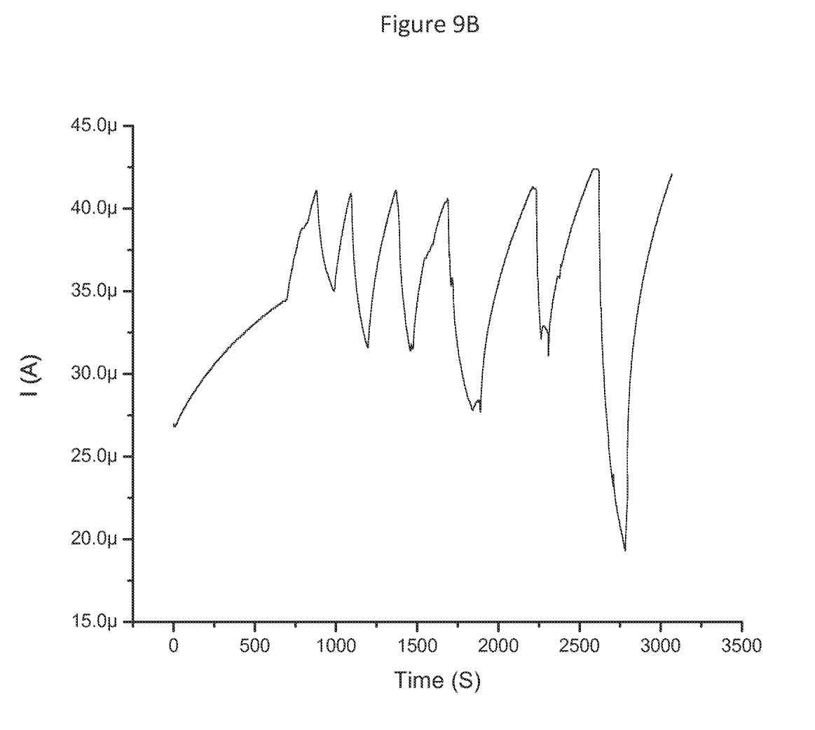

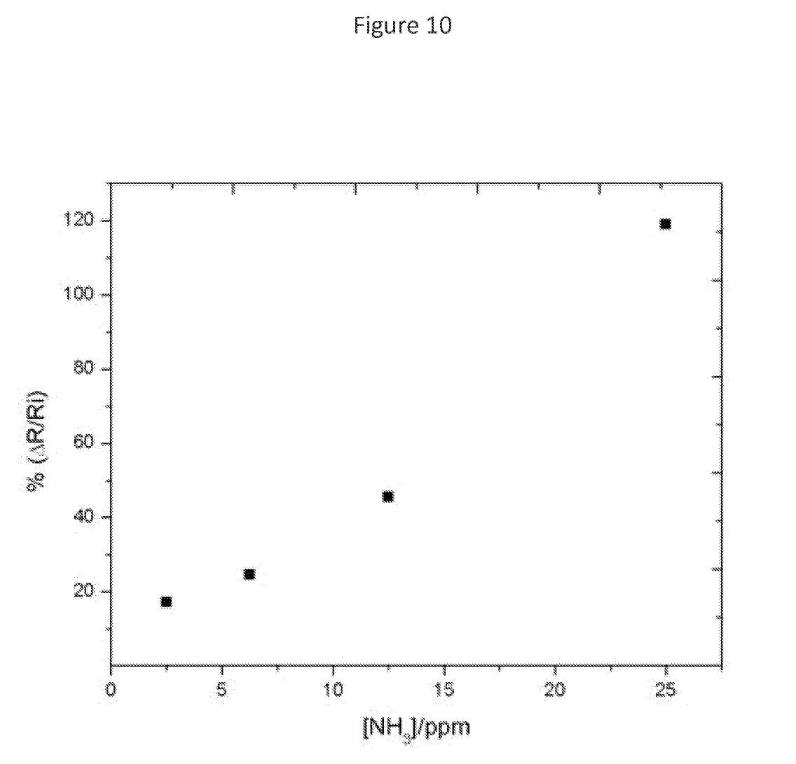

Example 6 was performed to determine the effect of ammonia on the sensor. First, a 5 L Tedlar bag including 25 ppm calibration ammonia was connected to an input of the gas diluter. The gas diluter diluted the ammonia with air to obtain samples with 25 ppm, 12.5 ppm, 2.5 ppm, 250 ppb and 25 ppb of ammonia and injected the diluted gas into the injection tube of the breath analyzer. At the same time, the resistivity of the sensor was measured for different dilution percentages. FIG. 9A shows the measured resistivity for 25 ppm, 12.5 ppm, 2.5 ppm, 250 ppb and 25 ppb of ammonia. FIG. 9B shows the corresponding change in the current. FIG. 10 also shows the response of the sensor plotted as the percentage change in the resistance (ΔR/Ri) vs. the concentration of ammonia, where ΔR=Rf−Ri (Ri being the initial resistance and Rf being the final or observed resistance on exposure to ammonia). These Figures illustrate that ammonia has a much higher effect on the resistivity of the sensor than carbon dioxide or dry nitrogen.

### EXAMPLE 7

### Determining Appropriate Volume of Breath Sample

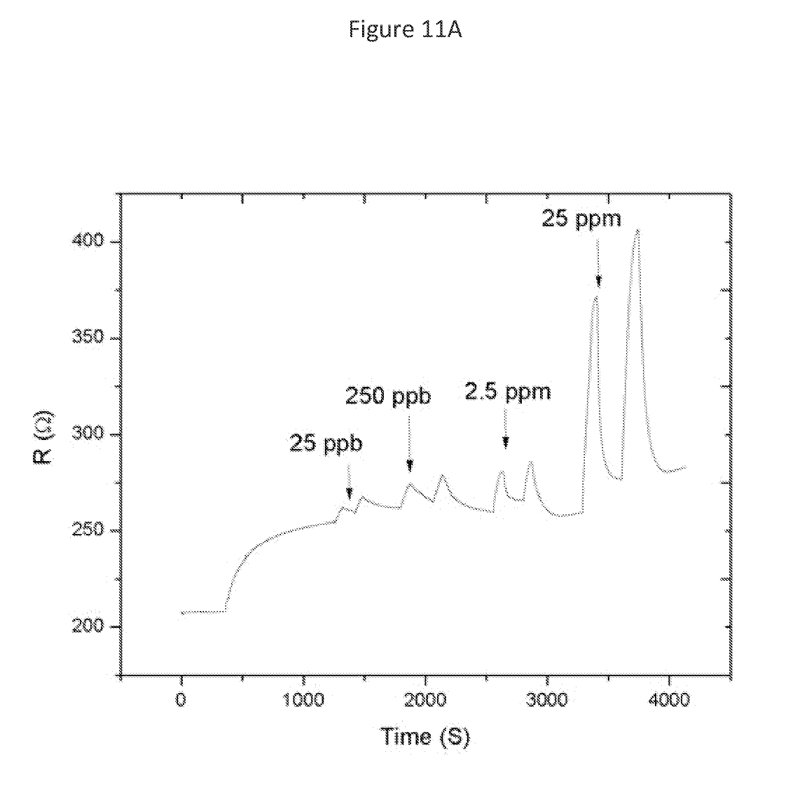

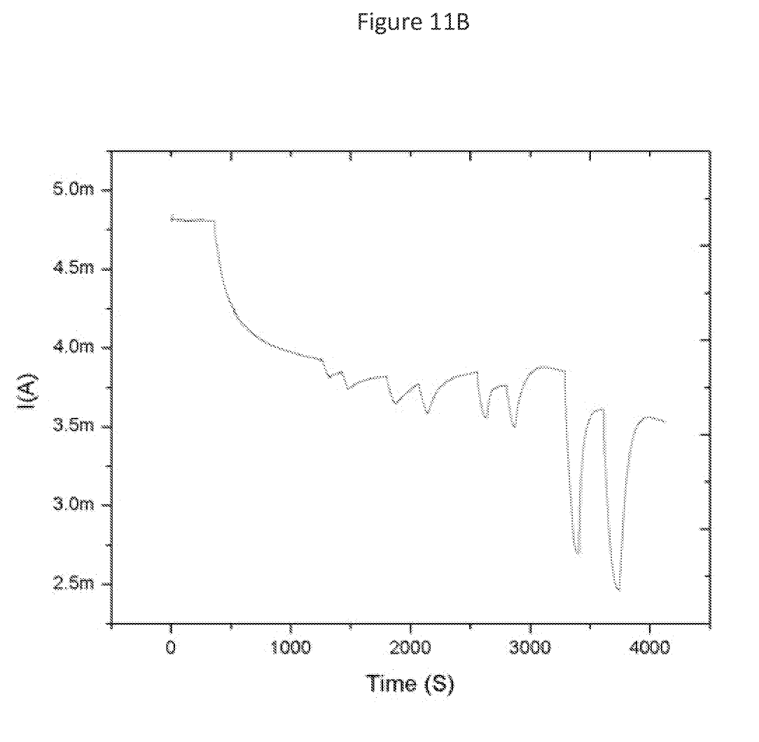

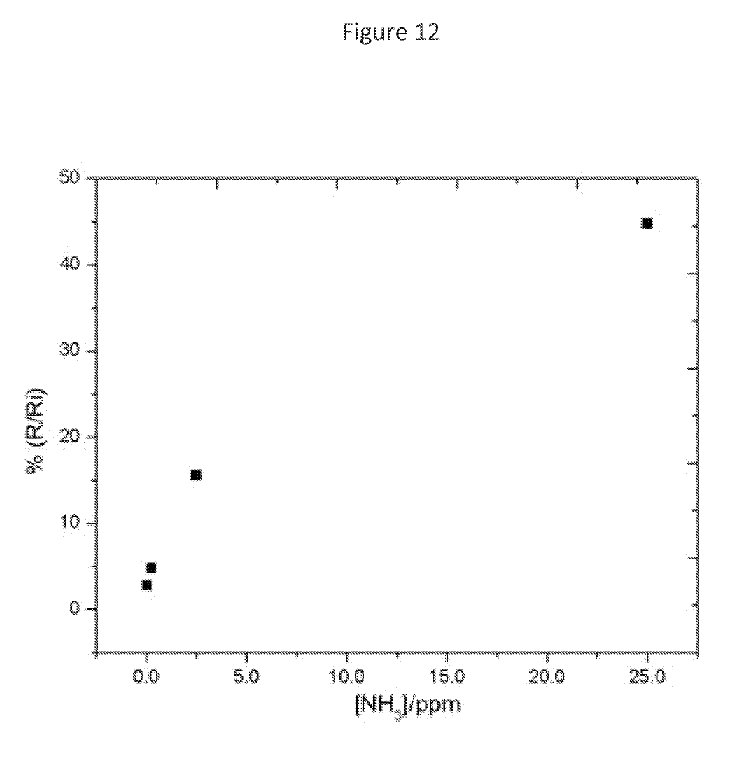

Example 7 was performed to determine an appropriate volume of breath sample. A technician first made serial dilutions of 25 ppm ammonia to 2.5 ppm to 250 ppb to 25 ppb in Tedlar bags. The technician then used a 20 cc syringe to collect samples from each Tedlar bag and inject them into the gas injection tube. At the same time, the technician measured the sensor resistivity. FIG. 11A shows the measured resistivity for 25 ppm, 2.5 ppm, 250 ppb and 25 ppb of ammonia. FIG. 11 B shows the corresponding change in the current. FIG. 12 also shows the response of the sensor plotted as the percentage change in the resistance (ΔR/Ri) vs. the concentration of ammonia, where ΔR=Rf−Ri (Ri being the initial resistance and Rf being the final or observed resistance on exposure to ammonia).

The syringe samples showed a lower percentage change in resistivity (45%) compared to the diluter samples (120%) at the 25 ppm concentration. This is because less volume of the gas was available to force a change in resistivity of the sensor. However, the percentage change at the 2.5 ppm concentration for the syringe samples was 15% compared to 17% for the gas diluter samples. This indicates that a breath sample volume of 20 cc is an effective volume to test breath samples. Also, the percentage change in resistivity observed for 250 ppb and 25 ppb was 4.8% and 2.5% respectively, which is a high enough response that allows breath samples in the trace level ppb values can be analyzed reliably.

### EXAMPLE 8

### Effect of Ammonia Compared to Carbon Dioxide and Dry Nitrogen

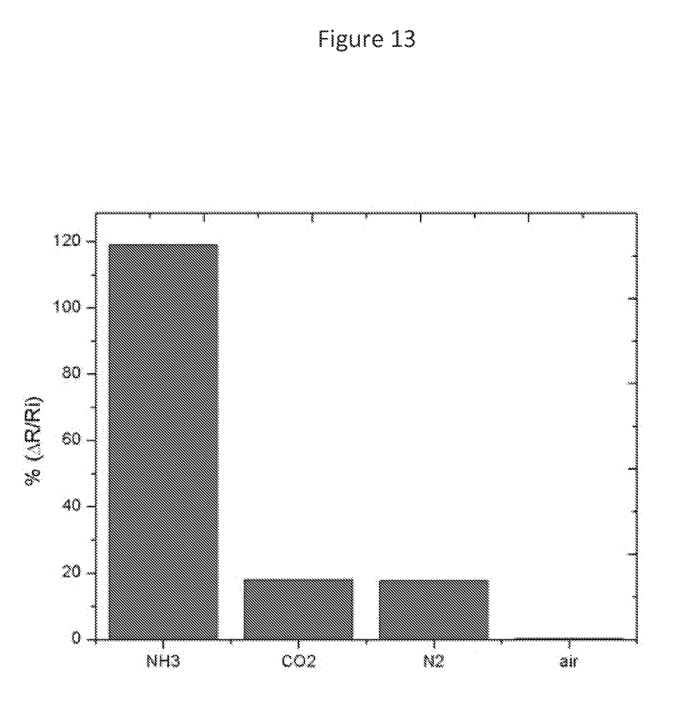

Example 8 was performed to determine the effect of ammonia compared to carbon dioxide and dry nitrogen. FIG. 13 shows a comparison of the response of 25 ppm ammonia (0.000025%) versus 100% dry nitrogen versus 100% carbon dioxide. The sensor responded much higher to ammonia compared to carbon dioxide and nitrogen. Thus, the sensor was considered effective in detecting ammonia in the trace ppb level.

### EXAMPLE 9

### Clinical Examples

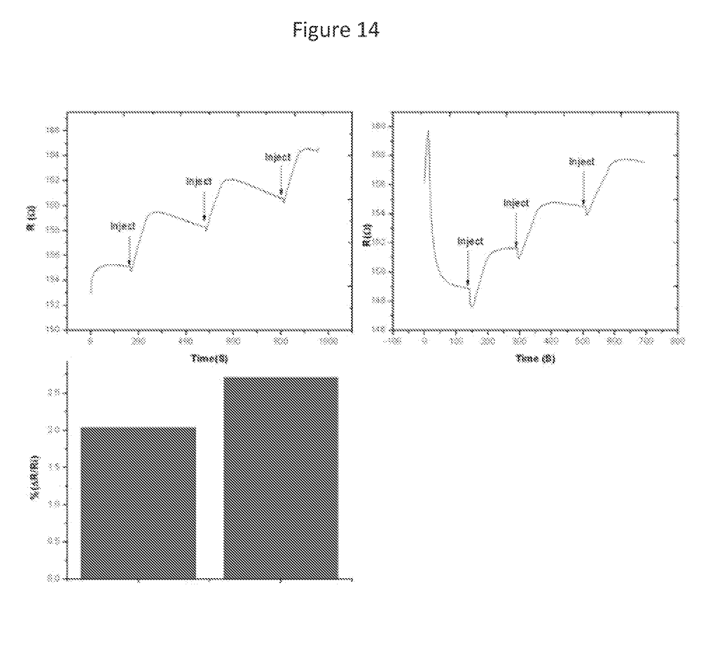

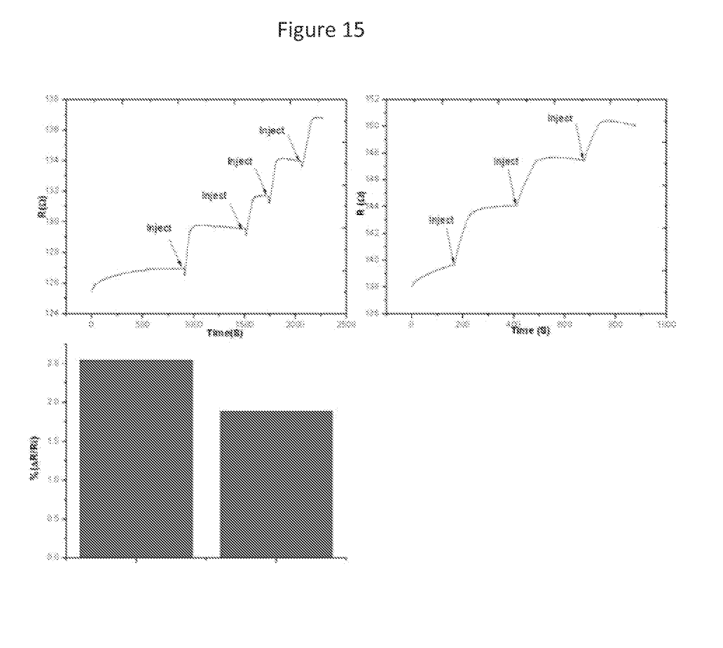

Example 9 includes several clinical testing examples. FIGS. 14-15 show results of these clinical testing examples. Two breath samples were collected from subjects recruited for the purpose of detecting presence of H. Pylori by measuring the amount of ammonia in their breath. The first sample was collected while the subject was fasting and was considered the baseline sample. The second sample was collected after the subject had ingested a urea-containing meal and was considered the post-urea ingestion sample.

A technician obtained these breath samples blindly and numbered in Tedlar bags. The technician collected all the breath samples from the Tedlar bags using a 20 cc syringe and then injected each syringe to deposit each sample into the injection tube for about 20 seconds and measured the resulting resistivity. The technician also plotted a change in resistivity as a function of ammonia concentration for each injection. Overall percentage change in resistivity was calculated as ΔR/Ri, where ΔR=Rf−Ri (Ri is the initial resistance and Rf the final or observed resistance on exposure to ammonia). In each calculation, the minimum resistance of each injection was considered for uniformity of the calculated resistivity change.

For each breath sample, the technician performed three injections. The technician then used the average value for the percentage change in plotting the bar plots. By using multiple injections, and then taking an average of the measurements, the technician obtained average values that were more apt to be accurate indicators of the ammonia values. FIGS. 14-15 show an increase in the amount of ammonia in breath detected between the baseline samples and the post urea ingestion samples, which indicates presence of H. Pylori in the subject's digestive tract.

Various examples of the invention have been described. Although the present invention has been described in considerable detail with reference to certain disclosed embodiments, the embodiments are presented for purposes of illustration and not limitation. Other embodiments incorporating the invention are possible. One skilled in the art will appreciate that various changes, adaptations, and modifications may be made without departing from the spirit of the invention and the scope of the appended claims.

What is claimed is:

1. A breath analyzer, comprising:
an input that receives a breath sample;
a sensor that contacts the breath sample, wherein the sensor comprises polyaniline and a conductive material, wherein the polyaniline contacts the conductive material, wherein the polyaniline is doped with a dopant that increases pH sensitivity of the polyaniline, and wherein the polyaniline has a resistivity that increases in response to increased concentration of ammonia;
a processor; and
an electrical circuit; wherein the electrical circuit operably connects the sensor to the processor, wherein the processor detects resistivity in the electrical circuit and uses the resistivity to calculate a concentration of ammonia in the breath sample.

2. The breath analyzer of claim 1 wherein the conductive material comprises a plurality of electrodes.

3. The breath analyzer of claim 2 wherein the plurality of electrodes include interdigitated finger electrodes.

4. The breath analyzer of claim 1 wherein the polyaniline has a pH sensitivity of more than 59 mV.

5. The breath analyzer of claim 1 wherein the dopant comprises a protonic acid.

6. The breath analyzer of claim 6 wherein the dopant comprises a protonic acid selected from the group consisting of hydrochloric acid, sulfuric acid, salicylic acid, acetic acid, citric acid, tartaric acid, oxalic acid, malonic acid, succinic acid, glutamic acid, adipic acid, phthalic acid and camphor sulfonic acid.

7. The breath analyzer of claim 1 wherein the analyzer further comprises a desiccant positioned between the input and the sensor.

8. The breath analyzer of claim 1 wherein the sensor detects ammonia at levels less than 50 ppb.

9. A handheld, portable breath analyzer, comprising:
a removable mouthpiece; and
a main body;
wherein the removable mouthpiece comprises a first portion and a second portion, wherein the first portion is sized and shaped to accommodate a user's lips and the second portion is sized and shaped to removably attach to the main body;
wherein the main body includes a sensor, a processor and an electrical circuit;
wherein the sensor comprises polyaniline, wherein the polyaniline is doped with a dopant that increases pH sensitivity of the polyaniline, and wherein the polyaniline has a resistivity that increases in response to increased concentration of ammonia;
wherein the electrical circuit operably connects the sensor to the processor; and
wherein the processor detects resistivity of the sensor and uses the resistivity to calculate a concentration of ammonia.

10. The breath analyzer of claim 9 wherein the removable mouthpiece further comprises one-way valve positioned between the first portion and the second portion, so that a breath sample can flow from the first portion to the second portion and cannot flow from the second portion to the first portion.

11. The breath analyzer of claim 9 further comprising a desiccant located in the second portion or in the main body.

12. The breath analyzer of claim 9 wherein the sensor is a replaceable sensor.

13. The breath analyzer of claim 9 wherein the polyaniline has a pH sensitivity of more than 59 mV.

14. The breath analyzer of claim 9 wherein the dopant comprises a protonic acid.

15. The breath analyzer of claim 14 wherein the dopant comprises a protonic acid selected from the group consisting of hydrochloric acid, sulfuric acid, salicylic acid, acetic acid, citric acid, tartaric acid, oxalic acid, malonic acid, succinic acid, glutamic acid, adipic acid, phthalic acid and camphor sulfonic acid.

16. A breath test method, comprising steps of:
(a) providing a portable breath analyzer, wherein the portable breath analyzer comprises:
(i) a removable mouthpiece; and
(ii) a main body, wherein the main body includes a sensor, a processor and an electrical circuit, wherein the sensor comprises ammonia selective material that has a resistivity that increases in response to increased concentration of ammonia, wherein the electrical circuit operably connects the sensor to the processor, and wherein the processor measure resistivity of the sensor;
(b) prompting a subject to exhale a baseline breath sample into the removable mouthpiece;
(c) allowing the processor to measure a resistivity of the sensor that occurs when the baseline breath sample contacts the sensor;
(d) prompting a subject to exhale a post-urea breath sample into the removable mouthpiece;
(e) allowing the processor to measure a resistivity of the sensor that occurs when the post-urea breath sample contacts the sensor; and
(f) comparing the measured resistivity of the baseline breath sample to the measured resistivity of the post-urea breath sample.

17. The breath test method of claim 16 further comprising resetting or replacing the sensor before the step of prompting a subject to exhale a post-urea breath sample into the removable mouthpiece.

18. A method of detecting presence of H. pylori in a digestive tract of a subject, the method comprising:
collecting a baseline breath sample from a subject;
determining an amount of ammonia present in the baseline breath sample;
collecting a post-urea breath sample from the subject;
determining an amount of ammonia present in the post-urea breath sample; and
designating a presence of H. pylori in the digestive tract if the amount of ammonia present in the post-urea breath sample exceeds the amount of ammonia present in the baseline breath sample by a predetermined value.

19. The method of claim 18 wherein the collecting a baseline breath sample from a subject and the collecting a post-urea breath sample from the subject comprises collecting both the baseline breath sample and the post-urea breath sample from a single portable breath analyzer.

20. The method of claim 18 wherein the determining an amount of ammonia present in the baseline breath sample comprises exposing the baseline breath sample to a sensor having a resistivity that increases in response to increased presence of ammonia and measuring resistivity of the baseline breath sample, and wherein the determining a concentration of ammonia present in the post-urea breath sample comprises exposing the post-urea breath sample to the sensor and measuring resistivity of the post-urea breath sample.

21. The method of claim 18 wherein the sensor comprises:
a conductive material;
a polyaniline in contact with the conductive material, wherein the polyaniline comprises polyaniline doped with a dopant that increases pH sensitivity of the polyaniline, and wherein the polyaniline has a resistivity that increases in response to increased presence of ammonia.

22. The method of claim 21 wherein the conductive material comprises a plurality of electrodes.

23. The method of claim 22 wherein the plurality of electrodes include interdigitated finger electrodes.

24. The method of claim 21 wherein the polyaniline has a pH sensitivity of more than 59 mV.

26. The method of claim 21 wherein the dopant comprises a protonic acid.

27. The method of claim 26 wherein the dopant comprises a protonic acid selected from the group consisting of hydrochloric acid, sulfuric acid, salicylic acid, acetic acid, citric acid, tartaric acid, oxalic acid, malonic acid, succinic acid, glutamic acid, adipic acid, phthalic acid and camphor sulfonic acid.
